(19) United States
(12) Patent Application Publication
(10) Pub. No.: US 2007/0026071 A1
(43) Pub. Date: Feb. 1, 2007

(54) Magnesium salt proton pump inhibitor dosage forms

(75) Inventors: Ranga R. Namburi, Robbinsville, NJ (US); Ravi Srikanth Tallapragada, Plainsboro, NJ (US); Subbaraju Gokaraju, Plainsboro, NJ (US); Burgise F. Palkhiwala, East Windsor, NJ (US)

Correspondence Address: Richard S. Roberts;Roberts & Roberts, L.L.P., Attorneys At Law, P.O. Box 484, Princeton, NJ, 08542-0484, US

(73) Assignee: QPharma, LLC

(21) Appl. No.: 11/191,520

(22) Filed: Jul. 28, 2005

(51) Int. Cl. A61K 9/14; A61K 31/4439

(52) U.S. Cl. 424/486; 424/488; 514/338

(57) ABSTRACT

The present invention concerns oral dosage formulations of sparingly to very slightly water soluble proton pump inhibitors, the oral dosage forms so made, and methods of use thereof. The oral dosage form has a core tablet of compressed particles composed of powder particles of a pharmaceutically acceptable material, having coated thereon admixture of a sparingly to very slightly water soluble magnesium salt of a benzimidazole proton pump inhibitor; and a hydrophilic polymer having a surfactant functionality that increases the water solubility of the magnesium salt of the benzimidazole proton pump inhibitor. The coated core tablet has a pharmaceutically acceptable sub-coating on the core tablet; and a pharmaceutically acceptable enteric coating on the sub-coating. The coated tablet may provide enhanced absorption when administered orally.

### BACKGROUND OF THE INVENTION

1. Field of the Invention

The present invention concerns oral dosage formulations of sparingly to very slightly water soluble proton pump inhibitors, the oral dosage forms so made, and methods of use thereof. More particularly, the invention concerns a pharmaceutical composition of a sparingly to very slightly water soluble magnesium salt of a benzimidazole proton pump inhibitor; and a hydrophilic polymer having a surfactant functionality that increases the water solubility of the magnesium salt of the benzimidazole proton pump inhibitor. Such compositions do not require organic solvents in their preparation.

2. Description of the Related Art

The proton pump, located in the apical membrane of the parietal cell, is responsible for the secretion of acid in the stomach when it is stimulated by the enzyme adenosine triphosphate (H⁺, K⁺)-ATPase. Proton pump inhibitors are a class of anti-secretory compounds used in the management of gastrointestinal disorders. They suppress gastric acid secretion by the specific inhibition of the (H⁺, K⁺)-ATPase enzyme system at the secretory surface of the gastric parietal cell.

A family of substituted benzimidazoles has been developed as specific proton pump inhibitors (PPIs). Thus, PPIs are well known in the art as gastric acid secretion inhibiting agents. Since the introduction of omeprazole (Prilosec™) in 1989, several other PPIs have become available that include Lansoprazole (Prevacid™), Rabeprazole (Aciphex™), Pantoprazole (Protonix™) and Esomeprazole (Nexium™). PPIs are inactivated by exposure to gastric juice and are delivered in delayed-release gelatin capsules containing enteric-coated granules (omeprazole and lansoprazole) or in delayed-release enteric-coated tablets (rabeprazole and patoprazole) or in delayed-release enteric-coated granules compressed in to tablet dosage forms (omeprazole, lansoprazole and esomeprazole). Also an intravenous form of pantoprazole is now available. U.S. Pat. No. 4,255,431 describes a compound 2-[2-(3,5-dimethyl-4-methoxy)-pyridyl methyl sulfinyl]-(5-methoxy)-benzimidazole (Omeprazole) or pharmaceutically acceptable salt or non-toxic acid addition salt as a therapeutic compound for mammals including man, suffering from gastric acid secretion disturbances. U.S. Pat. No. 4,628,098 discloses that Lansoprazole is a substituted benzimidazole 2-[[[3-methyl-4-(2,2,2-trifluroethoxy)-2-pyridyl]methyl]sulfinyl]benzimidazole, a compound and a pharmacologically acceptable salt thereof that inhibits gastric acid secretion. Omeprazole is useful as well for providing gastrointestinal cytoprotective effects in mammals and man. Omeprazole may be used for prevention and treatment of gastrointestinal inflammatory diseases including gastritis, gastric ulcer, and duodenal ulcer. Furthermore, omeprazole may be used for prevention and treatment of other gastrointestinal disorders where cytoprotective and/or gastric antisecretory effect is desirable, e.g. in patients with gastrinomas, acute upper gastrointestinal bleeding, and patients with a history of chronic and excessive alcohol consumption. Omeprazole is also known from U.S. Pat. Nos. 4,738,974; 4,786,505; 4,853,230; 5,690,960; 5,690,960; 5,714,504; 5,714,504; 5,877,192; 5,900,424; 6,147,103; 6,150,380; 6,166,213; 6,191,148; 6,369,085; 6,369,085; and 6,428,810, among others. Lansoprazole is known from U.S. Pat. Nos. 4,628,098; 4,689,333; 5,013,743; 5,026,560; 5,045,321; 5,093,132; 5,433,959; 5,464,632; 6,123,962; and 6,328,994, among others. Rabeprazole is known from U.S. Pat. Nos. 5,035,899 and 5,045,552. Pantoprazole is known from U.S. Pat. Nos. 4,758,579 and 5,997,903, among others.

U.S. Pat. No. 6,403,616 describes examples of dissolution rate from pharmaceutical dosage forms manufactured from different batches of omeprazole magnesium. Specifically the composition of Example 2 suspension uses omeprazole magnesium, hydroxy propyl methyl cellulose, adjusting the pH of the suspension and spray layering the suspension on to sugar spheres. Hydroxy propyl methyl cellulose at 15% by weight of active is used as a binder in the process. In the current invention the hydrophilic polymer hydroxy propyl methyl cellulose is used at a higher concentration considering the active and polymer ratio on weight by weight basis and at this ratio the polymer acts as a surfactant besides its use a binder. U.S. Pat. No. 5,900,424 describes omeprazole magnesium salt form with a crystallinity of not less than 75% and methods of making such forms. Magnesium salts of benzimidazoles are also known in U.S. Pat. Nos. 5,690,960; 5,753,265; and 6,428,810.

U.S. published patent application US20040052847 concerns methods of making oral formulations of drugs having an extremely low solubility in water by converting crystalline active compounds into an amorphous state during coating or spray coating of core particles.

Structurally PPIs contain a sulfinyl group bridging between substituted benzimidazole and pyridine rings. Once these compounds reach the parietal cells and diffuse into the secretory canaliculi, they become protonated. The protonated compounds rearrange to form sulfenic acid and then a sulfenamide. The latter interacts covalently with sulfhydryl groups at critical sites in the extracellular (luminal) domain of the membrane spanning (H⁺, K⁺)-ATPase. Inhibition occurs when two molecules of the inhibitor are bound per molecule of the enzyme. The specificity of these proton pump inhibitors arises from the selective distribution of the (H⁺, K⁺)-ATPase, the acid-catalyzed rearrangement of the compounds to generate the active inhibitor, and the trapping of the protonated compound and the cationic sulfenamide within the acidic canaliculi and adjacent to the target enzyme.

PPIs are typically administered orally as delayed-release dosage forms. The compounds are stable in alkaline pH but are destroyed by gastric acid. Therefore, if the integrity of the enteric coated micro granules or enteric coated non-spherical beads or enteric coated tablets is destroyed in any way and the patient swallows such enteric-coated dosage forms, the acidic pH in the stomach will break down the active compounds. The delayed release dosage forms, when appropriately taken, release the PPIs after the dosage forms leave the stomach.

A variety of adverse reactions have been ascribed to proton pump inhibitors, such as omeprazole and lansoprazole, although the incidence of adverse reactions is low, and the adverse reactions are generally minor. Due to the profound reduction in gastric acidity, there tends to be an increased secretion of gastrin. Hence, patients who take therapeutic doses of PPIs have modest hypergastrinemia. Prolonged administration of high doses of the drugs can cause hyperplasia of oxyntic mucosal cells.

The most common side effects of proton pump inhibitors, such as omeprazole and lansoprazole, are nausea, diarrhea, and abdominal colic. The drugs can also result in bacterial overgrowth in the gastrointestinal tract and the development of nosocomial pneumonia. Omeprazole however is only stable in basic pH conditions and degrades rapidly in acid pH environment and the rate of degradation of lansoprazole in aqueous solution increases with decreasing pH. The degradation half-life of lansoprazole in aqueous solution at 25° C. is approximately 0.5 hour at pH 5.0 and approximately 18 hours at pH 7.0. For this reason the omeprazole, lansoprazole and other PPI oral dosages form must be protected, not only from the pharmaceutical formulation ingredients acidic in nature used to make a dosage form but also from the acidic gastric fluid in order to reach the absorption site in the small intestine. Manufacturing processes currently employ lengthy enteric coating process times for providing complete gastric protection of drug loaded granules. Also sodium salt forms of rabeprazole and pantoprazole are formulated to provide better stability of these PPIs in tablet dosage forms. Conversion of these PPIs in to their respective salts require additional lengthy manufacturing processing step. Extrusion and spheronization process for producing multi unit particulates and or small spherical seeds layered with benzimidazole proton pump inhibitors and coating with protective sub-coating followed by enteric coating are the techniques employed in the currently manufactured drug products.

The percent bioavailability of omeprazole from commercially marketed omeprazole dosage forms is 30-40. Lansoprazole, Rabeprazole and Pantoprazole dosage forms provide 80-85%, 52% and 77% respectively of active drugs. Increased bioavailability from the dosage forms help to decrease the daily dose requirements.

Hence, there is a need in the art for proton pump inhibitors that have improved stability of dosage forms, ease in manufacturing techniques, enhanced oral absorption and better gastroprotective properties, decreased the recurrence of ulcers, facilitate ulcer healing and that can be used at low dosages. The present invention is directed to these, as well as other, important ends.

### SUMMARY OF THE INVENTION

The invention provides a pharmaceutical composition comprising:
a sparingly to very slightly water soluble magnesium salt of a benzimidazole proton pump inhibitor;
a pharmaceutically acceptable, water-soluble, hydrophilic polymer having a surfactant functionality that increases the water solubility of the sparingly to very slightly water soluble magnesium salt of the benzimidazole proton pump inhibitor; and
water.

The invention also provides a method of producing a pharmaceutical suspension which comprises admixing a sparingly to very slightly soluble magnesium salt of a benzimidazole proton pump inhibitor; and a pharmaceutically acceptable, water-soluble, hydrophilic polymer having a surfactant functionality; and water.

The composition further provides a pharmaceutically acceptable particle comprising powder particles comprised of a pharmaceutically acceptable material, said powder particles having spray coated thereon a dried composition formed by admixing a sparingly to very slightly water soluble magnesium salt of a benzimidazole proton pump inhibitor; and a pharmaceutically acceptable, water-soluble, hydrophilic polymer having a surfactant functionality that increases the water solubility of the sparingly to very slightly water soluble magnesium salt of the benzimidazole proton pump inhibitor; and water. The spray coating of a micromatrix of the hydrophilic polymer and the proton pump inhibitor increases the water solubility of the sparingly to very slightly soluble proton pump inhibitor due to its surfactant property.

The composition yet further provides an oral pharmaceutical dosage form comprising:
a core tablet of compressed particles, said compressed particles comprising:
powder particles comprised of a pharmaceutically acceptable material, said powder particles having spray coated thereon a dried composition formed by admixing a sparingly to very slightly water soluble magnesium salt of a benzimidazole proton pump inhibitor; and a pharmaceutically acceptable, water-soluble, hydrophilic polymer having a surfactant functionality that increases the water solubility of the sparingly water soluble magnesium salt of the benzimidazole proton pump inhibitor; and water;
a pharmaceutically acceptable sub-coating on the core tablet; and
a pharmaceutically acceptable enteric coating on the sub-coating.

The composition further provides a method of producing pharmaceutically acceptable oral dosage form comprising:
(a) forming a suspension comprising:
a sparingly to very slightly water soluble magnesium salt of a benzimidazole proton pump inhibitor;
a pharmaceutically acceptable, water-soluble, hydrophilic polymer having a surfactant functionality that increases the water solubility of the sparingly to very slightly water soluble magnesium salt of the benzimidazole proton pump inhibitor; and
water;
(b) coating the suspension from (a) onto powder particles comprised of a pharmaceutically acceptable material; combining said coated powder particles with a pharmaceutically acceptable disintegrating agent and a pharmaceutically acceptable lubricant;
(c) compressing the result from (b) into a core tablet;
(d) coating said core tablet with a pharmaceutically acceptable sub-coating composition; and
(e) applying a pharmaceutically acceptable enteric coating on the sub-coating.

### DETAILED DESCRIPTION OF THE INVENTION

One aspect of the invention concerns an admixture of water; a sparingly to very slightly water soluble magnesium salt of a benzimidazole proton pump inhibitor; and a pharmaceutically acceptable, water-soluble, hydrophilic polymer having a surfactant functionality that increases the water solubility of the sparingly to very slightly water soluble magnesium salt of the benzimidazole proton pump inhibitor. The spray coating process forms a micromatrix of polymer and active pharmaceutical ingredient and this micromatrix mechanism enhances the water solubility of the sparingly to very slightly water soluble magnesium salt of the benzimidazole proton pump inhibitor.

As used herein, the term “proton pump inhibitor” refers to any compound that reversibly or irreversibly blocks gastric acid secretion by inhibiting the H⁺/K⁺-ATP ase enzyme system at the secretory surface of the gastric parietal cell.

Useful proton pump inhibitors for use in the present invention non-exclusively include magnesium salts of benzimidazoles, for example, magnesium salts of substituted benzimidazoles and magnesium salts of substituted azabenzimidazoles, including, for example, magnesium salt of omeprazole, magnesium salt of lansoprazole, magnesium salt of pantoprazole, magnesium salt of rabeprazole, magnesium salt of leminoprazole, magnesium salt of timoprazole, magnesium salt of tenatoprazole, magnesium salt of disulprazole, magnesium salt of esomeprazole and combinations thereof. The magnesium salt may be in a crystalline form, an amorphous form, a hydrate form or an anhydrous form. Preferred are the magnesium salt of omeprazole and magnesium salt of esomeprazole. These are considered to be sparingly to very slightly water soluble magnesium salts. Sparingly soluble salts have a water solubility of 1 part by weight salt in from about 30 parts by weight to about 100 parts water and very slightly soluble salts have a water solubility of 1 part by weight salt in from about 1000 to about 10,000 parts by weight water.

In one embodiment of the coating suspension composition, the magnesium salt of the benzimidazole proton pump inhibitors may be present in the overall suspension composition in an amount of from about 0.1% w/v to about 20.0% w/w. In another embodiment, the magnesium salt of the benzimidazole proton pump inhibitors may be present in the overall suspension composition in an amount of from about 1.0% w/v to about 10.0% w/w. In yet another embodiment, the magnesium salt of the benzimidazole proton pump inhibitors may be present in the overall composition in an amount of from about 2.5% w/w to about 5.0% w/w.

The overall suspension composition further comprises water. In one embodiment, water may be present in the composition in an amount of from 0.5% w/w to about 98.0% w/w. In another embodiment water may be present in the over all suspension composition in an amount of from 5.0% w/w to about 95.0% w/w. In still another embodiment water may be present in the suspension composition in an amount of from 10% w/w to about 92.0% w/w.

In another less preferred embodiment, the suspension composition may optionally further comprise a pharmaceutically acceptable, volatilizable, organic solvent which is miscible with water. Useful solvents include alcohols such as methyl alcohol, ethyl alcohol, butyl alcohol, isopropyl alcohol; ketones such as acetone; polyhydric alcohols, glycerin, hexylene glycol, propylene glycol, polyethylene glycol, and combinations thereof. Any suitable acetone may be used to carry out the present invention, such as Pharmacopeial or USP grade acetone. Ethyl alcohol is a preferred solvent. Denatured ethyl alcohol could be used in place of pure ethyl alcohol. In one embodiment, the solvent may be present in the composition in an amount of from about 1.0% w/w to about 90.0% w/w. In another embodiment, the cosolvent may be present in the solution composition in an amount of from about 10.0% w/w to about 88.0% w/w. In still another embodiment the solvent may be present in the solution composition in an amount of from about 80.0% w/w to about 86.0% w/w.

The solution composition further comprises a pharmaceutically acceptable, water-soluble, hydrophilic polymer having surfactant functionality. Examples of suitable water soluble polymers include, but are not limited to, alkylcelluloses such as methylcellulose, hydroxyalkylcelluloses such as hydroxymethylcellulose, hydroxyethylcellulose, hydroxypropylcellulose and hydroxybutylcellulose; hydroxyalkyl alkylcelluloses such as hydroxyethyl methylcellulose and hydroxypropyl methylcellulose; carboxyalkylcelluloses such as carboxymethylcellulose; alkali metal salts of carboxyalkylcelluloses such as sodium carboxymethylcellulose; carboxyalkylalkylcelluloses such as carboxymethylethylcellulose; carboxyalkylcellulose esters; starches; pectins such as sodium carboxymethylamylopectin; chitin derivatives such as chitosan; polysaccharides such as alginic acid, alkali metal and ammonium salts thereof, carrageenans, galactomannans, traganth, agar-agar, gum arabicum, guar gum and xanthan gum; polyacrylic acids and salts thereof; polymethacrylic acids and salts thereof, including methacrylate copolymers polyvinylpyrrolidone, copolymers of polyvinylpyrrolidone with vinyl acetate; polyalkylene oxides such as polyethylene oxide and polypropylene oxide and copolymers of ethylene oxide and propylene oxide; dextrins and maltodextrins etc. Useful hydroxy propyl methyl cellulose, is manufactured by Aqualon, USA, Dow Chemical Industries, USA and also by Shin-Etsu Chemical Company, Japan, in 5 mPa·s or 3 mPa·s viscosity grades. In one embodiment, the hydrophilic polymer component may be present in the overall suspension composition in an amount of from about 1.0% (w/w) to about 20.0% (w/w). In another embodiment, the hydrophilic polymer component may be present in the overall composition in an amount of from about 2.0% (w/w) to about 15.0% w/w. In yet another embodiment, the hydrophilic polymer component may be present in the overall composition in an amount of from about 2.5% (w/w) to about 10.0% w/w. With respect to the magnesium salt, the hydrophilic polymer is present in an amount of at least about 25% by weight of the sparingly to very slightly water soluble magnesium salt of a benzimidazole proton pump inhibitor. Usually, the hydrophilic polymer is present in an amount of from about 25% to about 500% by weight of the sparingly to very slightly water soluble magnesium salt of a benzimidazole proton pump inhibitor. In most preferred embodiment the hydrophilic polymer is present at 100.0% to about 200.0% by weight of the sparingly to very slightly water soluble magnesium salt of a benzimidazole proton pump inhibitor.

The magnesium salt containing composition is then spray coated on a pharmaceutically acceptable material in powder form. This process results in formation of a micro matrix of polymer and active pharmaceutical ingredient and the micro matrix enhances the water solubility of sparingly to very slightly soluble magnesium salt of proton pump inhibitor. The top spray granulation process removes the water and solvent if present mostly and converts the powder particles in to compressible granules. The resultant granules are combined with tablet disintegrating agents and lubricants and then compressed into a core tablet. Core powder particles used herein may be of any suitable size, but typically have a mean diameter of from about 20 to 1000 micrometers, preferably from about 20 micrometers to about 200 micrometers. Examples include particles with a diameter of about 20 to 200 micrometers Preferred core powder particles have a diameter of from about 20 micrometers to about 200 micrometers. Size of particles can be determined in accordance with known techniques, such as described in the CRC Handbook, 64th edition, page F-i 14 and USP24/NF19, page 1969.

The core powder particles may be formed of any suitable pharmaceutically acceptable material. Examples of such materials are polymers e.g., plastic resins; inorganic substances, e.g., silica, glass, hydroxyapatite, salts (sodium or potassium chloride, calcium or magnesium carbonate) and the like; organic substances, e.g., activated carbon, acids (citric, fumaric, tartaric, ascorbic and the like acids), and saccharides and derivatives thereof. Particularly suitable materials are saccharides such as sugars, oligosaccharides, polysaccharides and their derivatives, for example, glucose, rhamnose, galactose, lactose, sucrose, mannitol, sorbitol, dextrin, maltodextrin, cellulose, microcrystalline cellulose, sodium carboxymethyl cellulose, starches (maize, rice, potato, wheat, tapioca) and the like saccharides.

Preferred as a core material for carrying out the present invention is microcrystalline cellulose particles. Useful microcrystalline cellulose in the form of powder particles is available as AVICEL™ from FMC Corporation.

Besides microcrystalline cellulose powder particles, a combination of microcrystalline cellulose and disintegrating agent particles such as croscarmelose sodium in this invention could be used for spray coating of these particles with polymer/active ingredient micro matrix.

Tablets can be produced by conventional tabletting techniques with conventional ingredients or excipients. The tablets are preferably formed from a composition comprising the particles described herein distributed in a mixture of a disintegrating agent and a diluent or filler. Suitable diluents include, but are not limited to, lactose, sucrose, dextrose, mannitol, sorbitol, starch, cellulose, calcium phosphate, microcrystalline cellulose such as AVICEL™ etc. Tablets may include a variety of other conventional ingredients, such as binders, buffering agents, lubricants, glidants, thickening agents, sweetening agents, plasticizers, flavors, pigments, preservatives, complexing and chelating agents, electrolytes or other active ingredients in amounts of up to about 10 percent by weight based on the weight of the compressed tablet.

Useful lubricants non-exclusively include magnesium stearate, talc, stearic acid, polyethylene glycol, glyceryl behenate, zinc stearate, and vegetable oil derivatives and may be present in an amount of from about 0.1% by weight to about 5.0 percent by weight based on the weight of the compressed tablet.

Useful disintegrating agents non-exclusively include but are not limited to, crospovidone, croscarmellose sodium, sodium starch glycolate, various grades of starch, polacrilin potassium and may be present in an amount of from about 0.2% by weight to about 10.0 percent by weight based on the weight of the compressed tablet.

The compressed tablet is then preferably provided with a sealing sub-coating to separate the compressed tablet from a subsequently applied enteric coating. The sealing sub-coating protects the tablet active ingredients from chemical interactions with the enteric coating dispersion ingredients and thereby rendering proton pump inhibitors not to undergo acid catalyzed chemical degradation or any other degradation process.

Useful compositions for this purpose are well known in the art and are generally commercially available. The protective sub-coating can be applied by a standard film coating procedure in a suitable coating machine using aqueous dispersions containing hydroxypropyl methylcellulose, polyethylene glycol, hydroxyethyl cellulose, hydroxypropyl cellusose, and polyvinylpyrrolidone. One useful sub-coating is OPADRY® which is commercially available from Colorcon of West Point, Pa.

The compressed, sub-coated tablet is then applied with an enteric coating to deter disintegration in the stomach and the enteric polymer starts dissolving as the dosage units leave the stomach of the subject. The enteric coat surrounds the core dosage form with a film which is hydrophobic at acidic pH values. Enteric coatings are well known in the art. Such materials can include polymers, plasticizers, and optional excipients.

Suitable polymers for the enteric coating of this invention are insoluble in acidic environments (e.g., gastric juice) but are soluble at pH 5.5 and upwards. Such polymers include cellulose acetate phthalate, methacrylate-base polymers, cellulose acetate trimellitate, hydroxypropyl methylcellulose acetate succinate, hydroxypropyl methylcellulose phthalate, polyvinyl acetate phthalate, anionic phthalate polymers based on methacrylic acid and methacrylic acid esters, and the like. These compounds are either used alone or in combination in an organic solvent. Generally, the polymers are dissolved in organic solvents before being used in a film coating process. For example, they include methyl alcohol, ethyl alcohol, ethyl alcohol/water, isopropyl alcohol, isopropyl alcohol/water, n-butyl alcohol, propylene glycol, ethyleneglycol monobutyl ether, acetone, acetone/isopropyl alcohol, and the like. Aqueous based polymeric dispersions are preferred for enteric coating applications in pharmaceutical industries. Suitable plasticizers impart sufficient tensile strength to the coating to prevent film cracking. Such plasticizers include triethyl citrate, dibutyl phthalate, polyethylene glycols, propylene glycol, diethylphthalate, acetyl triethyl citrate, and the like. The coating procedures are performed in a suitable coating machine. Omeprazole and lansoprazole delayed release dosage forms (in capsules) are official in United States Pharmacopoeia (USP 28-NF23). Assay and drug release tests for the invention tablets are performed following the USP procedures with appropriate modifications in the sample preparation. Purity and related substances test is also conducted to evaluate the quality of the invention dosage forms.

Useful enteric coatings are also commercially available. Such include SURETERIC® and ACRYL-EZE™, both of which are commercially available from Colorcon of West Point, Pa.

Drug release of the delayed release (enteric coated) tablets (invention) data in comparison to that of marketed tablet formulation indicate that the invention tablets have consistently higher dissolution results and also accelerated stability of the invention tablets prove that the composition and process used in the manufacturing of invention tablets produce superior quality drug product as compared to marketed formulation. Better dissolution may provide better absorption from the invention tablets of the actives.

Subjects afflicted with a disorder that may be treated with the oral dosage forms described herein include both human subjects and mammalian subjects such as dogs, cats and rabbits, etc. Disorders with which such subjects may be afflicted include those for which the proton pump inhibitor compounds described above are known to be effective in treating. The dosage of proton pump inhibitor compounds will vary depending on factors such as the disease and severity thereof, the age, weight and condition of the subject, etc., but in some embodiments is from about 5.0 milligrams per unit dosage form to about 80.0 milligrams per unit dosage form. The dosage form or forms may be administered to the subject at a single time or (more preferably) on multiple occasions over the day, and may be administered to the subjects under fed conditions, that is, simultaneously with food, or shortly before or after the subject has eaten so that the residence time of the dosage form in the subject's stomach is longer as compared to fasted conditions, or may be administered to the subject under fasted conditions, that is, without concurrent food administration so that the residence time of the dosage form in the subject's stomach is shorter as compared to fed conditions.

The following non-limiting examples serve to illustrate the invention.

### EXAMPLE 1

Omeprazole (as Magnesium Trihydrate) Delayed Release Tablets 20 mg

This example describes the preparation of an oral delayed release tablet dosage form of Omeprazole (as Magnesium Trihydrate).

Ingredients: Ingredients for the preparation of Omeprazole as Magnesium Trihydrate delayed release Tablets 20 mg of the invention are set forth in the Tables 1a-1d below.

TABLE 1a
Manufacturing Formula for 1500 core tablets:

| Per Unit | | Formula Wt - for 1500 |
|---|---|---|
| in mg | Ingredient | core tablets in g |
| 34.07 | Hydroxypropyl Methyl Cellulose(¹) | 51.1 |
| 22.71 | Omeprazole (Omeprazole Magnesium | 34.1 |
| (20 mg | Trihydrate) 22.71 mg of Magnesium | |
| base) | Salt is equivalent to 20 mg of base. | |
| 0.0 | Purified Water(²) | 644.8 |
| 250.82 | Microcrystalline Cellulose Powder(³) | 376.2 |
| 14.45 | Croscarmellose Sodium (intra | 21.7 |
| | granular) | |
| 14.45 | Croscarmellose Sodium (extra | 21.7 |
| | granular) | |
| 2.0 | Magnesium Stearate | 3.0 |
| 1.5 | Talc | 2.25 |
| 340.0 | Core Tablet Total Weight | 510.0 |

(¹)Hydroxy propyl methyl cellulose of 3 centipoises viscosity grade was used
(²)Removed during processing
(³)Avicel ® pH102 grade was used

TABLE 1b
Suspension Formula for 1500 core tablets:

| | | For 1500 core |
|---|---|---|
| Name of the Ingredient | % | tablets |
| Omeprazole Magnesium | 4.67 | 34.1 g |
| Trihydate | | |
| HydroxyPropylMethCell | 7.00 | 51.1 g |
| (3 cps viscosity grade) | | |
| Purified Water | 88.33 | 644.8 g |

Process: Purified Water 644.8 gm was taken in to a stainless steel container. Hydroxypropyl methyl cellulose (HPMC) 51.1 g was added under continued stirring. Upon further stirring HPMC goes in to solution. The drug Omeprazole Magnesium Trihydrate 34.1 g was added in to the above mixture under stirring. The stirring was continued until the entire drug was dispersed uniformly to form a fine suspension and the final suspension was passed through a 100 mesh sieve prior to spray coating step. The pH of the suspension—9.6

A STREA-1 fluid bed processor equipped with a top spray insert was used for spray coating of the suspension onto microcrystalline cellulose powder 376.1 g and croscarmelose sodium 21.7 g (intra granular portion). A spray rate of 2.0 to 2.5 g per minute was used. Drug loading was performed at a product bed temperature of 28-45° C. with an air volume of 60-70 cubic meters per hour and atomizing air pressure of 2.2 to 2.6 bar. The drug loaded particles were dried for an additional 15 minutes at a product bed temperature not exceeding 45° C. to obtain a proper loss on drying value of between 1.0 to 3.5 percent for the granules.

Drug loaded particles were combined with croscarmellose sodium 21.7 g (extra granular portion), magnesium stearate 3.0 g and talc 2.25 g by blending in an appropriate blender. The blended granules were compressed into a core tablet with an average weight of 340.0 mg using a tablet press with caplet shaped punch tooling (0.541×0.238″) at an average hardness of 12 kg/cm² with a thickness range of 5.3-5.4 mm.

TABLE 1c
Sub-coating: Ingredients

| | | Formula weight - |
|---|---|---|
| Per Unit | | 1500 sub-coated |
| in mg | Ingredient | tablets in g |
| 340.0 | Core Tablet | 510.0 |
| 6.8 | Opadry-03K19229(¹) (2% Weight | 10.2 |
| | gain) | |
| 0.0 | Purified Water(²) | 125.8 |
| 346.8 | Sub-Coated Tablet Total Weight | 520.2 |

(¹)Opadry ® is supplied by Colorcon
(²)Removed during the process

Process: The sub-coating material Opadry® at 7.5% was dispersed in purified water under constant stirring. The dispersion was sprayed using a coating pan (O'Hara 15″) with baffles and at an atomization air pressure of 20 PSI; at an air flow of 165 CFM, product bed temperature of 42-51° C. and until the tablets obtain a weight gain of 2.0%.

TABLE 1d
Delayed Release (Enteric) Coating: Ingredients

| | | Formula weight - |
|---|---|---|
| Per Unit | | 1500 enteric coated |
| in mg | Ingredient | tablets in g |
| 346.8 | Sub-coated Tablet | 520.2 |
| 31.2 | AcrylEze-93F19255(¹) (9% | 46.8 |
| | Weight gain) | |
| 0.0 | Purified Water(²) | 187.2 |
| 378.0 | Final Enteric Coated Tablet | 567.0 |

(¹)AcrylEze ® is supplied by Colorcon
(²)Removed during the process

Process: 20.0 percent by weight suspension in water of Acryl-Eze (commercially available from Colorcon of West Point Pa.) was sprayed using a coating pan (O'Hara 15″) with baffles and at an atomization air pressure of 20 PSI; at an air flow of 165 CFM, product bed temperature of 42-51° C. and until the tablets obtain a weight gain of 9.0%.

### EXAMPLE 2

Esomeprazole (as Magnesium Dihydrate) Delayed Release Tablets 20 mg

This example describes the preparation of an oral delayed release tablet dosage form of Esomeprazole (as Magnesium Dihydrate).

Ingredients: Ingredients for the preparation of Esomeprazole as Magnesium Dihydrate delayed release Tablets 20 mg of the invention are set forth in the Tables 2a-2d below.

TABLE 2a
Manufacturing Formula for 1500 core tablets:

| | | Formula Wt - for |
|---|---|---|
| Per Unit | | 1500 core tablets |
| in mg | Ingredient | in g |
| 33.00 | Hydroxypropyl Methyl | 49.5 |
| | Cellulose(¹) | |
| 22.00 | Esomeprazole (Esomeprazole | 33.0 |
| (20 mg | Magnesium Dihydrate) | |
| base) | 22.0 mg of Magnesium Salt is | |
| | equivalent to 20 mg of base. | |
| 0.0 | Purified Water(²) | 647.5 |
| 252.6 | Microcrystalline Cellulose | 378.9 |
| | Powder(³) | |
| 14.45 | Croscarmellose Sodium (intra | 21.7 |
| | granular) | |
| 14.45 | Croscarmellose Sodium (extra | 21.7 |
| | granular) | |
| 2.0 | Magnesium Stearate | 3.0 |
| 1.5 | Talc | 2.25 |
| 340.0 | Core Tablet Total Weight | 510.0 |

(¹)Hydroxy propyl methyl cellulose of 3 centipoises viscosity grade was used
(²)Removed during processing
(³)Avicel ® pH102 grade was used

TABLE 2b
Suspension Formula for 1500 core tablets:

| | | For 1500 core |
|---|---|---|
| Name of the Ingredient | % | tablets |
| Esomeprazole | 4.52 | 33.0 g |
| Magnesium Dihydate | | |
| HydroxyPropylMethCell | 6.78 | 49.5 g |
| (3 cps viscosity grade) | | |
| Purified Water | 88.7 | 647.5 g |

Process: Purified water 647.5 gm was taken into a stainless steel container. Hydroxypropyl methyl cellulose (HPMC) 49.5 g was added under continued stirring. Upon further stirring HPMC goes into solution. The drug Esomeprazole Magnesium Dihydrate 33.0 g was added into the above mixture under stirring. The stirring was continued until the entire drug was dispersed uniformly to form a fine suspension and the final suspension was passed through a 100 mesh sieve prior to spray coating step. The pH of the suspension—9.88

A STREA-1 fluid bed processor equipped with a top spray insert was used for spray coating of the suspension onto microcrystalline cellulose powder 378.9 g and croscarmelose sodium 21.7 g (intra granular portion). A spray rate of 2.0 to 2.5 g per minute was used. Drug loading was performed at a product bed temperature of 28-45° C. with an air volume of 60-70 cubic meters per hour and atomizing air pressure of 2.2 to 2.6 bar. The drug loaded particles were dried for an additional 15 minutes at a product bed temperature not exceeding 45° C. to obtain a proper loss on drying value of between 1.0 to 3.5 percent for the granules.

Drug loaded particles were combined with croscarmellose sodium 21.7 g (extra granular portion), magnesium stearate 3.0 g and talc 2.25 g by blending in an appropriate blender. The blended granules were compressed into a core tablet with an average weight of 340.0 mg using a tablet press with caplet shaped punch tooling (0.541×0.238″) at an average hardness of 12 kg/cm² with a thickness range of 5.3-5.4 mm.

TABLE 2c
Sub-coating: Ingredients

| | | Formula weight - |
|---|---|---|
| Per Unit | | 1500 sub-coated |
| in mg | Ingredient | tablets in g |
| 340.0 | Core Tablet | 510.0 |
| 6.8 | Opadry-03K19229(¹) (2% Weight | 10.2 |
| | gain) | |
| 0.0 | Purified Water(²) | 125.8 |
| 346.8 | Sub-Coated Tablet Total Weight | 520.2 |

(¹)Opadry ® is supplied by Colorcon
(²)Removed during the process

Process: The sub-coating material Opadry® at 7.5% was dispersed in purified water under constant stirring. The dispersion was sprayed using a coating pan (O'Hara 15″) with baffles and at an atomization air pressure of 20 PSI; at an air flow of 165 CFM, product bed temperature of 42-51° C. and until the tablets obtain a weight gain of 2.0%.

TABLE 2d
Delayed Release (Enteric) Coating: Ingredients

| | | Formula weight - |
|---|---|---|
| Per Unit | | 1500 enteric coated |
| in mg | Ingredient | tablets in g |
| 346.8 | Sub-coated Tablet | 520.2 |
| 31.2 | AcrylEze-93F19255(¹) (9% | 46.8 |
| | Weight gain) | |
| 0.0 | Purified Water(²) | 187.2 |
| 378.0 | Final Enteric Coated Tablet | 567.0 |

(¹)AcrylEze ® is supplied by Colorcon
(²)Removed during the process

Process: 20.0 percent by weight suspension in water of Acryl-Eze (commercially available from Colorcon of West Point Pa.) was sprayed using a coating pan (O'Hara 15″) with baffles and at an atomization air pressure of 20 PSI; at an air flow of 165 CFM, product bed temperature of 42-51° C. and until the tablets obtain a weight gain of 9.0%.

### EXAMPLE 3

Phase Solubility Study in pH 6.8 Buffer (Simulated Intestinal Fluid) and Purified Water:

In this study the solubility of Omeprazole Magnesium Trihydrate and Esomeprazole Magnesium Dihydrate active materials in pH 6.8 buffer and purified water was performed. Also their solubility when present in tablet dosage forms prepared by direct compression technique (compositions as described in Tables 3 and 4) and through fluid bed technique process following examples of this invention was determined (Tables 1a-1d and 2a-2d) and results are tabulated in Table 5. A pH 6.8 buffer is prepared as described under the monograph of Omeprazole Delayed Release capsules for dissolution test in United States Pharmacopoeia. Purified water and pH 6.8 buffer were used at ambient temperature (approx. 22° C.).

TABLE 3
Omeprazole Magnesium Tablets:
Core formula of tablets compressed by direct blending process:

| | | Formula Wt - for |
|---|---|---|
| Per Unit | | 1000 core tablets |
| in mg | Ingredient | in g |
| 22.7 | Omeprazole (Omeprazole | 22.7 |
| (20 mg | Magnesium Trihydrate) | |
| base) | 22.7 mg of Magnesium Salt is | |
| | equivalent to 20 mg of base. | |
| 284.8 | Microcrystalline Cellulose | 284.8 |
| | Powder(³) | |
| 29.0 | Croscarmellose Sodium (extra | 29.0 |
| | granular) | |
| 2.0 | Magnesium Stearate | 2.0 |
| 1.5 | Talc | 1.5 |
| 340.0 | Core Tablet Total Weight | 340.0 |

TABLE 4
Esomeprazole Magnesium Tablets:
Core formula of tablets compressed by direct blending process:

| | | Formula Wt - for |
|---|---|---|
| Per Unit | | 1000 core tablets |
| in mg | Ingredient | in g |
| 22.0 | Esomeprazole (Esomeprazole | 22.0 |
| (20 mg | Magnesium Dihydrate) | |
| base) | 22.0 mg of Magnesium Salt is | |
| | equivalent to 20 mg of base. | |
| 285.5 | Microcrystalline Cellulose | 285.5 |
| | Powder(³) | |
| 29.0 | Croscarmellose Sodium (extra | 29.0 |
| | granular) | |
| 2.0 | Magnesium Stearate | 2.0 |
| 1.5 | Talc | 1.5 |
| 340.0 | Core Tablet Total Weight | 340.0 |

Solubility Study Procedure:

Samples of actives and tablet dosage form powders were used in the study. Each sample contains approx. 200 mg base equivalent Omeprazole and Esomeprazole as salts and such samples were transferred in to a centrifuge tube containing 20 mL of appropriate medium. Centrifuge tubes containing these suspensions were shaken for approx. 30 minutes on a wrist action shaker and samples were initially filtered through Whatman filter paper and subsequently filtered through 0.45 micron PTFE cartridge filters. The samples were further diluted by pipetting 1.0 mL in to 100 mL volumetric flasks and made up to the volume with appropriate medium. The optical density values were measured using a suitable UV-Visible spectrophotometer at 275 nm wavelength.

The quantities of actives dissolved from these samples were calculated and tabulated in the Table 5 below.

TABLE 5
Solubility

| | Active content | Active content |
|---|---|---|
| | equivalent to base | equivalent to base |
| Sample Identification | in pH 6.8 buffer | in purified water |
| Omeprazole Magnesium | 0.31 mg/mL | 0.26 mg/mL |
| Trihydrate Active Material | | |
| Omeprazole Magnesium | 0.36 mg/mL | 1.23 mg/mL |
| Trihydrate Direct Compression | | |
| Formula Tablets | | |
| Omeprazole Magnesium | 1.23 mg/mL | 2.56 mg/mL |
| Trihydrate Fluid Bed process | | |
| Tablets (Example 1) | | |
| Esomeprazole Magnesium | 1.17 mg/mL | 5.02 mg/mL |
| Dihydrate Active Material | | |
| Esomeprazole Magnesium | 1.16 mg/mL | 5.90 mg/mL |
| Dihydrate Direct Compression | | |
| Formula Tablets | | |
| Esomeprazole Magnesium | 1.67 mg/mL | 8.15 mg/mL |
| Dihydrate Fluid Bed process | | |
| Tablets (Example 2) | | |

The data from this study clearly indicates there is increased solubility of Magnesium salts of Omeprazole and Esomeprazole Actives when processed through fluid bed process as described in Examples 1 and 2. There is approx. 10 fold increased solubility of Omeprazole Active in purified water and 4 fold increased solubility of Omeprazole active in pH 6.8 buffer (simulated intestinal fluid) by the fluid bed process as compared to the active when tested alone. Esomeprazole as magnesium dihydrate is also increased by approx. 1.6 fold in water when processed in combination with hydroxy propyl methyl cellulose through fluid bed coating process. The spray coating process in presence of a water soluble hydrophilic polymer increases the solubility of actives both in water and in simulated intestinal gastric fluid through formation of micromatrix of polymer and active pharmaceutical ingredient.

### EXAMPLE 4

Tablets of Example 1 according to the invention were compared with marketed tablet dosage forms (Prilose® OTC 20 mg) for their acid resistance in 0.1N HCl exposed for 2 hours. All dissolution parameters maintained as per the monograph under Omeprazole delayed release capsules in USP 28 using paddle method, and the RPM of the paddles was set at 75.

TABLE 6
Omeprazole (as Magnesium) Drug Release comparison:
Acid Resistance Stage
Example 1 Tablets vs. Prilosec ® OTC 20 mg
Conditions: USP Type 2 (Paddles, RPM 75, Volume 500 mL)
Medium Used: 0.1N Hydrochloric Acid
% Drug Release in 0.1N Hydrochloric Acid after 2 hour

| | Example 1 Tablets | Prilosec ® OTC 20 mg |
|---|---|---|
| Unit No. | Per Unit | Per Unit |
| Unit 1 | 1.4 | 0.9 |
| Unit 2 | 0.8 | 1.1 |
| Unit 3 | 1.5 | 2.1 |
| Unit 4 | 1.1 | 1.8 |
| Unit 5 | 1.3 | 1.5 |
| Unit 6 | 0.7 | 1.4 |

Tablets of the invention prepared according to Example were compared with marketed tablet dosage forms for their dissolution in the pH 6.8 phosphate buffer after exposing them for two hours in 0.1N hydrochloric acid for 2 hours. All dissolution parameters maintained as per the monograph under Omeprazole delayed release capsules in USP 28 using paddle method, while setting the RPM at 75.

TABLE 7
Omeprazole (as Magnesium) Drug Release comparison:
Buffer Stage
Example 1 tablets vs. Prilosec ® OTC 20 mg tablets
Conditions: USP Type 2 (Paddles, RPM 75, Volume 900 mL)
Medium Used: 0.1N Hydrochloric Acid for 2 hours followed by pH 6.8 Phosphate buffer

| Time | | % Quantity of Omeprazole dissolved | | | | | | | |
|---|---|---|---|---|---|---|---|---|---|
| Interval | Sample | 1 | 2 | 3 | 4 | 5 | 6 | Min. | Max. |
| 15 min | Example 1 | 68.9 | 71.7 | 80.6 | 76.4 | 79.7 | 74.1 | 68.9 | 80.6 |
| | Tablets | | | | | | | | |
| | Prilosec ® OTC | 53.3 | 47.2 | 61.5 | 56.9 | 51.4 | 49.2 | 47.2 | 61.5 |
| | 20 mg Tablets | | | | | | | | |
| 30 min | Example 1 | 98.4 | 99.0 | 98.1 | 97.5 | 99.1 | 97.9 | 97.5 | 99.1 |
| | Tablets | | | | | | | | |
| | Prilosec ® OTC | 94.6 | 96.7 | 95.3 | 96.4 | 94.9 | 96.1 | 94.6 | 96.7 |
| | 20 mg Tablets | | | | | | | | |

### EXAMPLE 5

Tablets of the invention prepared according to Example 2 are tested for their acid resistance in 0.1N HCl exposed for 2 hours. All dissolution parameters maintained as per the monograph under Omeprazole delayed release capsules in USP 28 using paddle method, while setting up the RPM to 75.

TABLE 8
Esomeprazole (as Magnesium) Drug Release:
Acid Resistance Stage
Invention Tablets
Conditions: USP Type 2 (Paddles, RPM 75, Volume 500 mL)
Medium Used: 0.1N Hydrochloric Acid
% Drug Release in 0.1N Hydrochloric Acid after 2 hours

| | Invention Tablets |
|---|---|
| Unit No. | Per Unit |
| Unit 1 | 0.7 |
| Unit 2 | 0.6 |
| Unit 3 | 1.1 |
| Unit 4 | 0.9 |
| Unit 5 | 1.2 |
| Unit 6 | 1.0 |

Tablets of the invention prepared according to Example 2 were tested for their dissolution in the pH 6.8 phosphate buffer after exposing them for two hours in 0.1N hydrochloric acid for 2 hours. All dissolution parameters maintained as per the monograph under Omeprazole delayed release capsules in USP 28 using paddle method, while setting up the RPM at 75.

TABLE 9
Esomeprazole Mg Drug Release as Esomeprazole:
Buffer Stage
Example 2 tablets
Conditions: USP Type 2 (Paddles, RPM 75, Volume 900 mL)
Medium Used: 0.1N Hydrochloric Acid for 2 hours followed by pH 6.8 Phosphate buffer

| Time | | % Quantity of Omeprazole dissolved | | | | | | | |
|---|---|---|---|---|---|---|---|---|---|
| Interval | Sample | 1 | 2 | 3 | 4 | 5 | 6 | Min. | Max. |
| 15 min | Example 2 | 82.3 | 84.6 | 81.2 | 79.6 | 81.9 | 83.6 | 79.6 | 84.6 |
| | Tablets | | | | | | | | |
| 30 min | Example 2 | 98.4 | 96.2 | 99.1 | 97.5 | 97.9 | 98.5 | 96.2 | 99.1 |
| | Tablets | | | | | | | | |

### EXAMPLE 6

The enteric tablets obtained according to Examples 1 and 2 and a reference sample were stored at 40° C. and 75% RH for eight weeks and the appearance of each tablet core and microgranules in the case of marketed sample were observed. The physical observation results were tabulated in Table 10.

TABLE 10
Accelerated Conditions of 40° C. and 75% RH

| Sample | | | | | |
|---|---|---|---|---|---|
| details | 1^{st} Week | 2^{nd} Week | 4^{th} Week | 6^{th} Week | 8^{th} Week |
| Example 1 | − | − | − | − | − |
| tablets | | | | | |
| Example 2 | − | − | − | − | − |
| tablets | | | | | |
| Reference | − | − | − | ± | ± |
| Example | | | | | |

Note:
− not changed (white)
± somewhat changed

Invention tablets have better physical stability when placed under accelerated stability conditions.

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

What is claimed is:

1. A pharmaceutical composition comprising:
a sparingly to very slightly water soluble magnesium salt of a benzimidazole proton pump inhibitor;
a pharmaceutically acceptable, water-soluble, hydrophilic polymer having a surfactant functionality that increases the water solubility of the sparingly to very slightly water soluble magnesium salt of the benzimidazole proton pump inhibitor; and
water.

2. The composition of claim 1 wherein the magnesium salt is in a crystalline form, an amorphous form, or a hydrate form.

3. The composition of claim 1 wherein the a sparingly to very slightly water soluble magnesium salt of a benzimidazole proton pump inhibitor has a water solubility of 1 part by weight salt in from about 30 parts by weight water to about 10,000 parts by weight water.

4. The composition of claim 1 wherein the hydrophilic polymer is present in an amount of at least about 25% by weight of the sparingly to very slightly water soluble magnesium salt of a benzimidazole proton pump inhibitor.

5. The composition of claim 1 wherein the hydrophilic polymer is present in an amount of from about 25% to about 500% by weight of the sparingly to very slightly water soluble magnesium salt of a benzimidazole proton pump inhibitor.

6. The composition of claim 1 wherein the sparingly to very slightly soluble magnesium salt of a benzimidazole proton pump inhibitor comprises a magnesium salt of omeprazole, pantoprazole, rabeprazole, leminoprazole, lansoprazole, timoprazole, tenatoprazole, disulprazole, esomeprazole, and combinations thereof.

7. The composition of claim 1 wherein the sparingly to very slightly soluble magnesium salt of a benzimidazole proton pump inhibitor comprises a magnesium salt of omeprazole.

8. The composition of claim 1 wherein the sparingly to very slightly soluble magnesium salt of a benzimidazole proton pump inhibitor comprises a magnesium salt of esomeprazole.

9. The composition of claim 1 wherein the pharmaceutically acceptable, water-soluble, hydrophilic polymer having a surfactant functionality is selected from the group consisting of alkylcelluloses, hydroxyalkylcelluloses; hydroxyalkyl alkylcelluloses, carboxyalkylcelluloses; alkali metal salts of carboxyalkylcelluloses; carboxyalkylalkylcelluloses; carboxyalkylcellulose esters; starches; pectins; chitin derivatives; polysaccharides; polyacrylic acids and salts thereof; polymethacrylic acids and salts thereof, polyvinylpyrrolidone, copolymers of polyvinylpyrrolidone with vinyl acetate; polyalkylene oxides; dextrins and maltodextrins.

10. The composition of claim 1 wherein the pharmaceutically acceptable, water-soluble, hydrophilic polymer having a surfactant functionality comprises hydroxypropyl methyl cellulose.

11. The composition of claim 1 wherein the sparingly to very slightly soluble magnesium salt of a benzimidazole proton pump inhibitor comprises a magnesium salt of omeprazole, a magnesium salt of esomeprazole, or combinations thereof; the pharmaceutically acceptable, water-soluble, hydrophilic polymer having a surfactant functionality comprises hydroxypropyl methyl cellulose; and wherein the hydrophilic polymer is present in an amount of from about 25% to about 500% by weight of the sparingly to very slightly water soluble magnesium salt of a benzimidazole proton pump inhibitor.

12. A method of producing a pharmaceutical suspension which comprises admixing water; a sparingly to very slightly soluble magnesium salt of a benzimidazole proton pump inhibitor; and a pharmaceutically acceptable, water-soluble, hydrophilic polymer having a surfactant functionality.

13. The method of claim 12 wherein the sparingly to very slightly soluble magnesium salt of a benzimidazole proton pump inhibitor comprises a magnesium salt of omeprazole, a magnesium salt of esomeprazole, or combinations thereof; the pharmaceutically acceptable, water-soluble, hydrophilic polymer having a surfactant functionality comprises hydroxypropyl methyl cellulose;

14. A pharmaceutically acceptable particle comprising powder particles comprised of a pharmaceutically acceptable material, said powder particles having spray coated thereon a dried composition formed by admixing, a sparingly to very slightly water soluble magnesium salt of a benzimidazole proton pump inhibitor; and a pharmaceutically acceptable, water-soluble, hydrophilic polymer having a surfactant functionality that increases the water solubility of the sparingly to very slightly water soluble magnesium salt of the benzimidazole proton pump inhibitor; and water.

15. The pharmaceutically acceptable particle of claim 14 wherein the sparingly to very slightly soluble magnesium salt of a benzimidazole proton pump inhibitor comprises a magnesium salt of omeprazole, a magnesium salt of esomeprazole, or combinations thereof; the pharmaceutically acceptable, water-soluble, hydrophilic polymer having a surfactant functionality comprises hydroxypropyl methyl cellulose.

16. The pharmaceutically acceptable particle of claim 14 wherein the powder particle has a mean diameter of from about 20 micrometers to about 200 micrometers.

17. The pharmaceutically acceptable particle of claim 14 wherein the powder particle comprises microcrystalline cellulose and/or croscarmelose sodium.

18. An oral pharmaceutical dosage form comprising:
a core tablet of compressed particles, said compressed particles comprising:
powder particles comprised of a pharmaceutically acceptable material, said powder particles having spray coated thereon a dried composition formed by admixing a sparingly to very slightly water soluble magnesium salt of a benzimidazole proton pump inhibitor; and a pharmaceutically acceptable, water-soluble hydrophilic polymer having a surfactant functionality that increases the water solubility of the sparingly water soluble magnesium salt of the benzimidazole proton pump inhibitor; and water;
a pharmaceutically acceptable sub-coating on the core tablet; and
a pharmaceutically acceptable enteric coating on the sub-coating.

19. The pharmaceutical oral dosage form of claim 18 wherein the sparingly to very slightly soluble magnesium salt of a benzimidazole proton pump inhibitor comprises a magnesium salt of omeprazole, a magnesium salt of esomeprazole, or combinations thereof; and the pharmaceutically acceptable, water-soluble, hydrophilic polymer having a surfactant functionality comprises hydroxypropyl methyl cellulose.

20. The oral dosage form of claim 18 wherein said core tablet of compressed particles further comprises at least one pharmaceutically acceptable disintegrating agent and pharmaceutically acceptable lubricant.

21. The pharmaceutical oral dosage form of claim 18 further comprising microcrystalline cellulose powder, croscarmellose sodium, magnesium stearate and talc.

22. A method of treating a disorder in a subject in need thereof, comprising orally administering to said subject an oral dosage form according to claim 18 in a pharmaceutically acceptable amount.

23. A method of producing pharmaceutically acceptable oral dosage form comprising:
(a) forming a suspension comprising:
a sparingly to very slightly water soluble magnesium salt of a benzimidazole proton pump inhibitor;
a pharmaceutically acceptable, water-soluble, hydrophilic polymer having a surfactant functionality that increases the water solubility of the sparingly to very slightly water soluble magnesium salt of the benzimidazole proton pump inhibitor; and
water;
(b) coating the suspension from (a) onto powder particles comprised of a pharmaceutically acceptable material; combining said coated powder particles with a pharmaceutically acceptable disintegrating agent and a pharmaceutically acceptable lubricant;
(c) compressing the result from (b) into a core tablet;
(d) coating said core tablet with a pharmaceutically acceptable sub-coating composition; and
(e) applying a pharmaceutically acceptable enteric coating on the sub-coating.

24. The method of claim 23 wherein the sparingly to very slightly soluble magnesium salt of a benzimidazole proton pump inhibitor comprises a magnesium salt of omeprazole, a magnesium salt of esomeprazole, or combinations thereof; and the pharmaceutically acceptable, water-soluble, hydrophilic polymer having a surfactant functionality comprises hydroxypropyl methyl cellulose.

25. The method of claim 23 wherein step (b) coating comprises spray.
